(19) United States
(12) Patent Application Publication
(10) Pub. No.: US 2016/0101253 A1
(43) Pub. Date: Apr. 14, 2016

(54) PROTECTIVE ENDOTRACHEAL TUBE

(71) Applicant: Husam Ibrahim ALAHMADI, Atlanta, GA (US)

(72) Inventor: Husam Ibrahim ALAHMADI, Atlanta, GA (US)

(21) Appl. No.: 14/509,845

(22) Filed: Oct. 8, 2014

(51) Int. Cl. A61M 16/04

(52) U.S. Cl. CPC A61M 16/0445; A61M 16/0425; A61M 16/0463; A61M 16/0486; A61M 16/044

(57) ABSTRACT

Embodiments include a tracheal tube having a proximal end and a distal end. In certain embodiments, a plurality of spaced-apart cuffs disposed around the tracheal tube. The plurality of spaced-apart cuffs being configured to seal the tracheal airway of a patient and to capture tracheal secretions or debris. Also, at least two pilot balloons are connected to the plurality of cuffs, the at least two pilot balloons being configured to inflate and deflate the plurality of cuffs. Further, at least two injection lumens connected to at least two injection distal ports configured to administer fluids to the bronchi of the patient and at least one evacuation lumen connected to a plurality of evacuation ports, the at least one evacuation lumen being connected to a vacuum source. The plurality of evacuation ports are disposed proximal the plurality of evacuation ports to remove the captured fluids or debris.

### GRANT OF NON-EXCLUSIVE RIGHT

This application was prepared with financial support from the Saudia Arabian Cultural Mission, and in consideration therefore the present inventor(s) has granted The Kingdom of Saudi Arabia a non-exclusive right to practice the present invention.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

When treating a patient, a tracheal tube, for example, endotracheal, endobronchial, nasotracheal, or transtracheal device may be used to control the flow of gases into the trachea of a patient. Often, a seal or cuff between the outside of the tube and the interior wall of the tracheal lumen is required, allowing for the generation of positive intrathoracic pressure distal to the seal and prevention of ingress of solid or liquid matter into the lungs from proximal to the seal.

Endotracheal tubes (ETT) are used in medical procedures, such as endotracheal intubation. Endotracheal intubation is the placement of a flexible tube into the trachea (windpipe) to maintain an open airway. The ETT is inserted from the mouth (orotracheal) or from the nose (nasotracheal). The ETT may be used in both humans and animals where patients have either stopped breathing on their own or are in need of anesthetic gases in their lungs.

Generally, endotracheal tubes are made from soft biomaterials, which are hard to manipulate. A professional caretaker must insert the ETT with great care. An endotracheal tube would normally have a cuff that is used to seal the airway and minimize aspiration.

Conventionally, a single cuff is inflated with air following insertion of the device into the human mid-trachea to achieve an airtight seal of the space between the tube and surrounding tracheal wall. As such the cuffed endotracheal tube has been routinely employed for many decades to prevent upper airway obstruction or to facilitate artificial ventilation of the unconscious or anesthetized patient. However, recent investigations have disclosed several defects in conventional cuffed endotracheal tubes. These defects are, firstly, the failure to prevent secretions from accumulating in the upper trachea. Secondly, all tracheal tubes traverse the delicate structures of the larynx and abrade the vocal cords as a result of to and fro motion of the tube associated with respiration. Thirdly, the effects of intra-cuff sealing pressure upon the ciliated membranes lining the tracheal wall reversibly or permanently injure the cilia and surface membranes of the mid-trachea. The degree of injury is proportional to the magnitude of lateral-wall-cuff pressure in excess of 15 centimeters (cm) of water and to the duration such pressure is applied. Conventionally, the inflatable cuff is placed to rest in the mid-trachea several centimeters below the larynx, where compression of the ciliated endothelium of the trachea causes injury. Conventional endotracheal tube (ETT) cuffs have a single cavity and produce a non-leak seal at pressures which occlude the blood perfusion of the tracheal mucosa and after a period of time produce tissue necrosis.

One problem arising from prevailing practices of tracheal intubation or the insertion of the ETT is the failure of the conventional cuff to prevent secretions from passing through an unprotected space between the vocal cords and the endotracheal tube. The result is an accumulation of a ring of contaminated material in the upper trachea above the inflated cuff which enters the lung when the cuff is deflated at extubation or the removal of the ETT. During intubation this residue or ring of infected secretions may trickle into the larynx and become entrapped above the inflated cuff until subsequent extubation allows the ring of secretions to enter the mid-trachea where injured cilia fail to protect the lung. The normal protective mechanisms by which the cilia carry the secretions upward in the respiratory tree until reflex coughing results in their removal fail to operate.

### SUMMARY

Embodiments include an endotracheal tube (ETT) having a tracheal tube having a proximal end and a distal end. The ETT also includes a plurality of spaced-apart cuffs disposed around the tracheal tube, the plurality of spaced-apart cuffs being configured to seal a tracheal airway of a patient and to capture tracheal secretions or debris. The ETT further includes at least two pilot balloons connected to the plurality of spaced-apart cuffs, the at least two pilot balloons being configured to inflate and deflate the plurality of spaced-apart cuffs. The ETT also includes at least two injection lumens connected to at least two injection distal ports and being configured to administer fluids to the bronchi of the patient. The ETT further includes at least one evacuation lumen connected to a plurality of evacuation ports, the at least one evacuation lumen being connected to a vacuum source. The plurality of evacuation ports is disposed proximal the plurality of spaced-apart cuffs to remove the captured fluids or debris.

Embodiments also include an endotracheal tube (ETT) having means for intubating a patient via their trachea. The ETT also includes means for sealing a tracheal airway of the patient and for capturing tracheal secretions or debris within the patient. The ETT further includes for inflating and deflating the means for sealing and capturing. The ETT also includes means for administering fluids to the bronchi of the patient. The ETT further includes means for evacuating the captured tracheal secretions or debris. The means for evacuating is disposed proximal the means for capturing tracheal secretions or debris.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIGS. 1A and 1B are perspective views of an endotracheal tube (ETT) apparatus according to certain embodiments of the disclosure.

FIG. 2 is an enlarged view of a first evacuation port and a first cuff of an ETT apparatus according to certain embodiments of the disclosure.

FIG. 3 is an enlarged view of a second evacuation port and a second cuff of an ETT apparatus according to certain embodiments of the disclosure.

FIG. 4 is an illustrative view of an ETT apparatus inserted via the mouth and through the trachea of a patient according to certain embodiments of the disclosure.

### DETAILED DESCRIPTION OF THE EMBODIMENTS

Referring now to the drawings, wherein like reference numerals designate identical or corresponding parts throughout the several views.

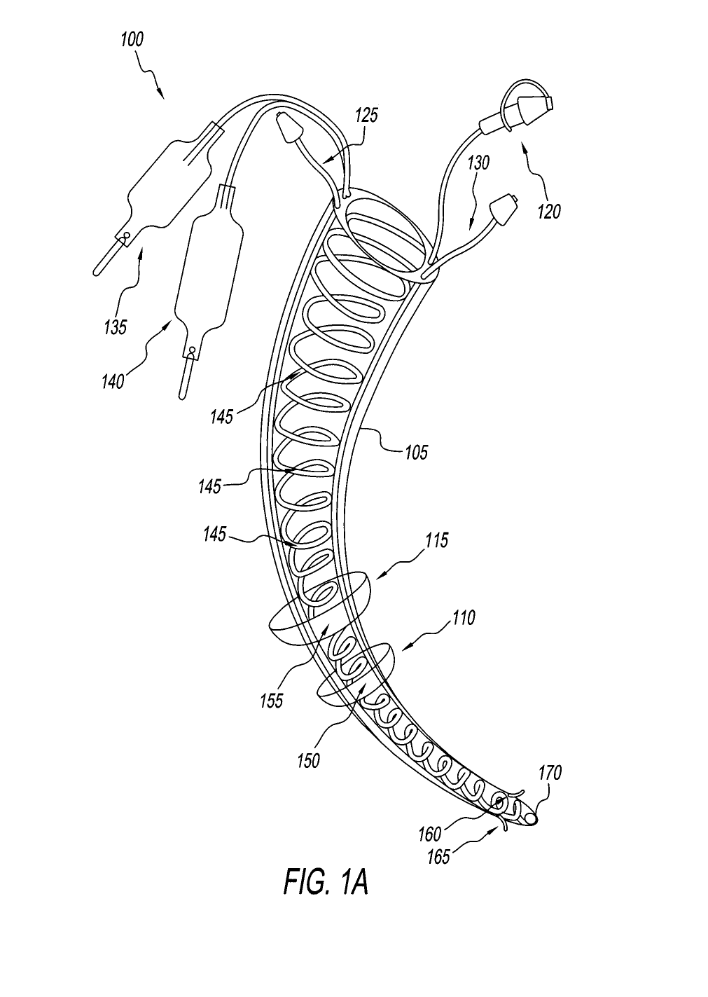

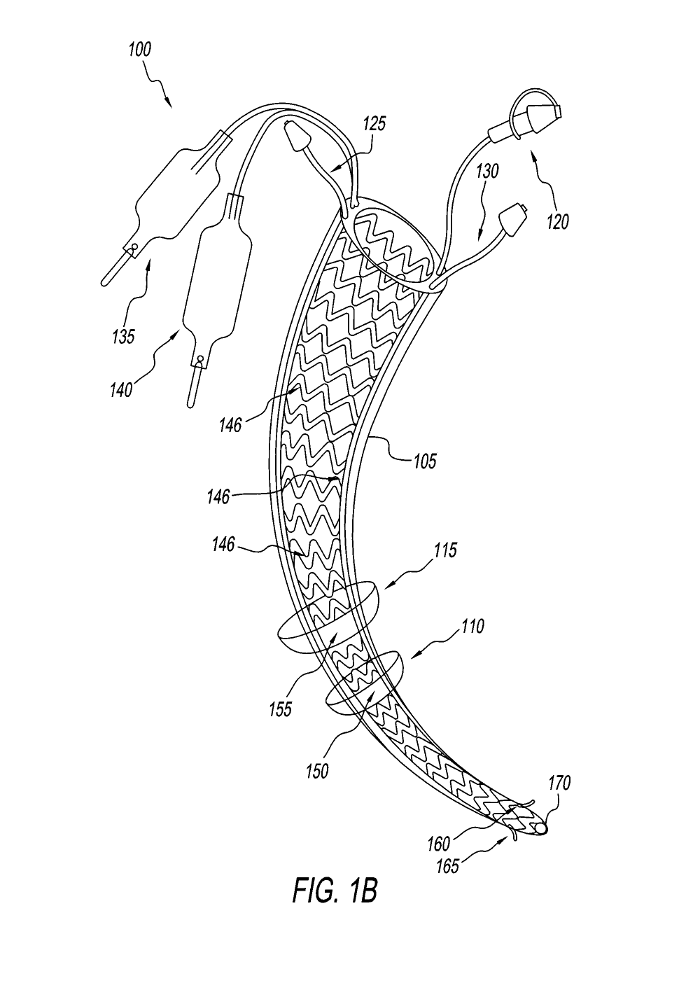

FIGS. 1A and 1B are perspective views of an endotracheal tube (ETT) apparatus 100 according to certain embodiments of the disclosure. In FIG. 1A, the ETT 100 includes a tracheal tube 105, a first pilot balloon 135, a second pilot balloon 140, a first injection lumen 125, a second injection lumen 130, an evacuation lumen 120, internal lining 145, a first cuff 110, a second cuff 115, a first evacuation port 150, a second evacuation port 155, a first injection distal port 160, a second injection distal port 165, and a beveled opening 170.

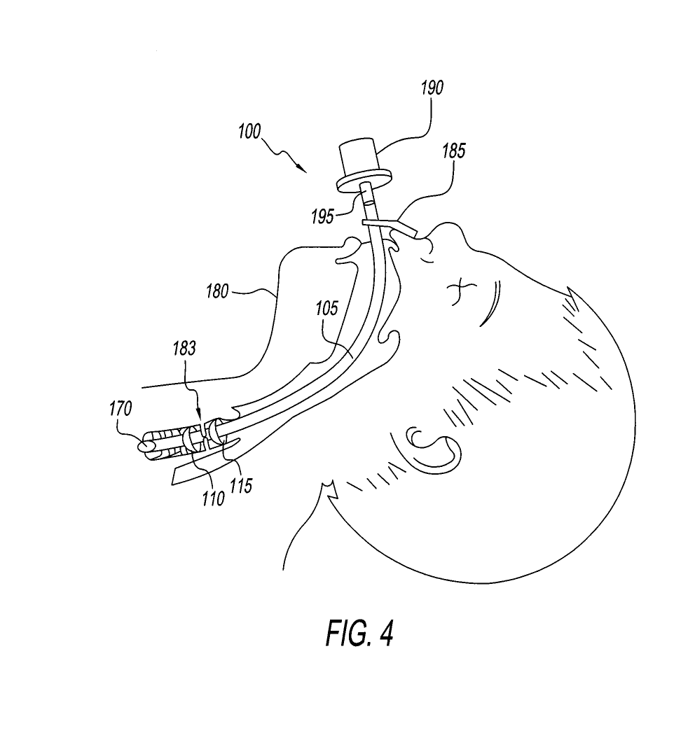

Tracheal tube 105 may be configured to taper from a proximal end 195 (near a patient's mouth/nose) down to the beveled opening 170 (see FIG. 4). The internal lining 145 may be configured to be disposed on the inner wall of tracheal tube 105 to help maintain and reinforce the form and shape of the tracheal tube 105 during use. In certain embodiments, the internal lining 145 is composed of copper and the internal lining 145 may be configured to extend the length of the tracheal tube 105. Copper surfaces have intrinsic properties which tend to destroy a wide range of microorganisms. Further, copper and its alloys (e.g., brasses, bronzes, cupronickel, copper-nickel-zinc, and others) are natural antimicrobial materials. Accordingly, in other embodiments, combinations of materials and alloys may be used. Thus, in this copper example, the internal lining 145 interrupts or suppresses the growth of pathogens that may harbor disease delivered to the patient's lungs, thereby reducing the occurrence of post-operative lung infection.

As shown in FIGS. 1A and 1B, the internal lining 145, 146 may also be configured to support the structure of the tracheal tube 105 by providing strength and helping maintain patency of the tracheal tube 105 before and during use. In other words, the internal lining 145, 146 may be formed as a spiral at 145, helical, or sinuous shape at 146 extending the length of the tracheal tube 105 in order to provide better support for the tracheal tube 105 and thereby prevent kinks, bends, or collapse of tracheal tube 105 while at the same time requiring less material for the internal lining 145.

The first pilot balloon 135 and the second pilot balloon 140 are configured to connect via a channel within the tube wall of the tracheal tube 105 to the first cuff 110 and the second cuff 115, respectively, in order to inflate or deflate each cuff during extubation or intubation. The pilot balloons 135, 140 may also be used to assess the amount of air pressure present in each cuff to prevent damage to the walls or cilia of the trachea during use. In other words, the first cuff 110 and the second cuff 115 may be configured to be inflated (pressurized) symmetrically (equally) via pilot balloons 135, 140, respectively. Such a pressurized configuration may facilitate sealing the airway with the least amount of pressure, thereby further protecting the tracheal wall from tracheal hypoperfusion due to asymmetrical cuff-inflation that could lead to tracheal necrosis. Hypoperfusion may occur if the intra-cuff pressure exceeds the perfusion pressure in a patient.

By having two pilot balloons 130, 135 connected to two cuffs 110, 115, an increase in safety for the ETT 100 may be achieved. For instance, the availability of two cuffs 110, 115 and two intact pilot balloons 130, 135 may eliminate the need to replace the ETT 100 in the case one of the pilot balloons rupturing, or a lumen is accidentally cut which connects the cuffs 110, 115 to the pilot balloons 135, 140. The remaining pilot balloon and cuff may be sufficient protection from aspiration for the patient by still providing the patient with a closed system with the mechanical ventilator and therefore his/her airway would be protected from aspiration. In other words, by having two cuffs 110, 115, the ETT 100 is more securely sealed and held in place within the patient than by a conventional single cuff endotracheal tube configuration, and the two cuffs 110, 115 increase the capture of excess fluids/leakage into the trachea.

The first injection lumen 125 and the second injection lumen 130 may be configured to independently supply a patient with any needed medications or anesthetics while using the ETT 100. The first and second injection lumens 125, 130 are configured to connect via channels within the tube wall of the tracheal tube 105 to the first injection distal port 160 and the second injection distal port 165, respectively. The first and second injection distal ports 160, 165 are disposed proximal to the beveled opening 170. The beveled opening 170 is disposed at the tapered end of the tracheal tube 180. The injection distal ports 160, 165 are disposed at or near the beveled opening 170 of the tracheal tube 105. Each port 160, 165 may be directed towards a bronchus to enable administering chemical fluids, such as a sedation agent or paralytic agent, directly into the right and/or the left bronchus simultaneously or in an independent manner.

The two injection lumens 125, 130 are configured to provide the capability of administering fluids directly to a single bronchus and/or both bronchi of the patient. The evacuating lumen 120 may be disposed at the side of the ETT, connecting the evacuation ports 150, 155 to a vacuum source (not shown) and may be used to remove all fluids collected by the evacuation ports 150, 155.

The evacuation ports 150, 155 may be configured to have a circumferential or 360° opening or port in order to more effectively and efficiently suction or capture any and all tracheal fluids or debris, thereby preventing aspiration of the patient. Such a configuration to suction fluids or debris in a patient's trachea from entering the lungs may prevent medical problems going forward, such as, ventilator-associated pneumonia (VAP) by more than 75%. The evacuation ports 150, 155 may be configured to be of various sizes to provide varying degrees of suction and fluid/debris removal.

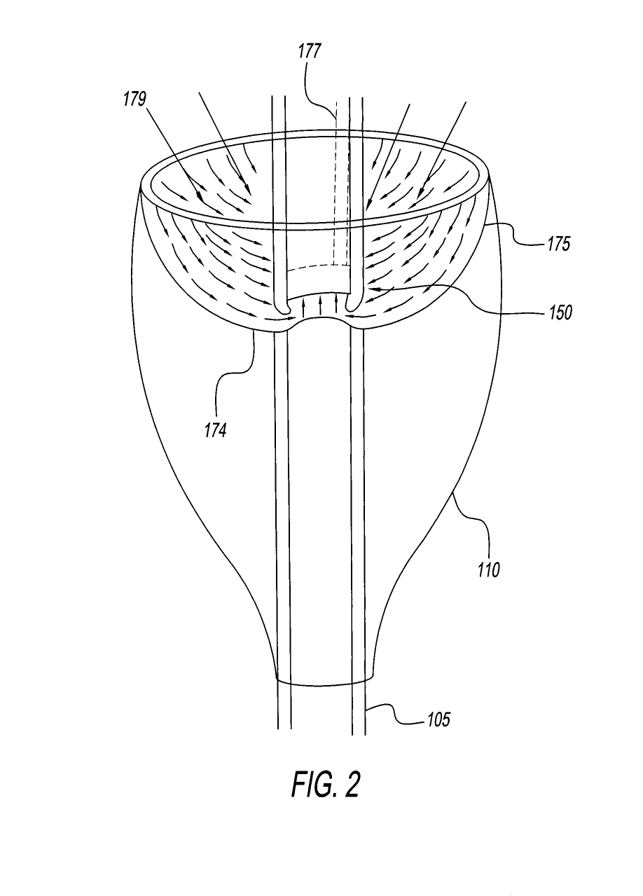

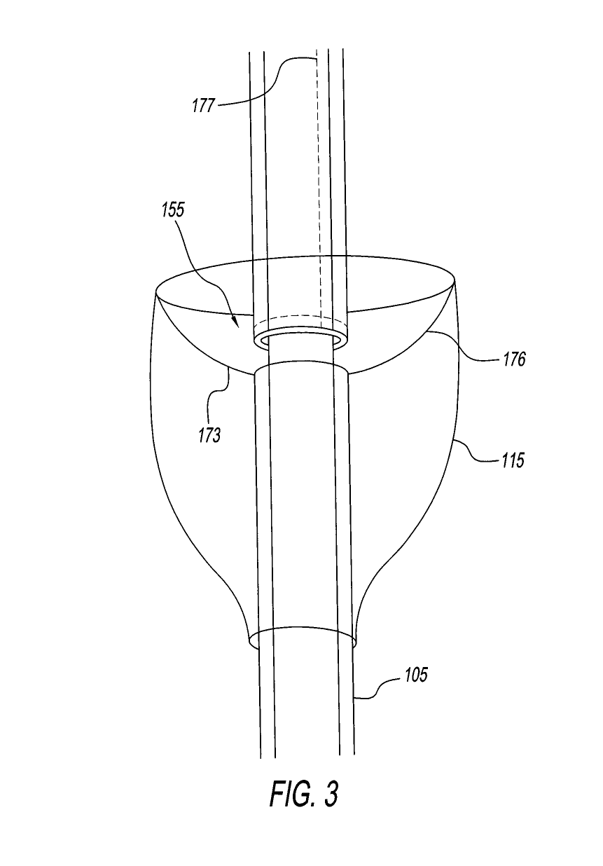

The evacuation lumen 120 may be configured to suction fluids or debris while using the ETT 100. The evacuation lumen 120 is configured to connect at one end via an evacuation channel 177 (see FIG. 2) connected to each cuff 110, 115. The evacuation lumen 120 is also configured to be connected at an opposite end to a suction or vacuum source (not shown) for collection purposes. Evacuation ports 150, 155 may be configured to be circumferential or 360° suction/drainage ports about tracheal tube 105 and disposed at the bottom of inward funnel-shaped portions 175, 176 (see FIGS. 2 and 3) of the cuffs 110, 115, respectively. Evacuation ports 150, 155 are configured to capture and suction any fluids coming down from the trachea. The ETT 100 may be configured with larger-sized circumferential or 360° evacuation ports 150, 155 disposed at the bottom of each of the cuffs 110, 115. Such a configuration may assure complete drainage/suction of fluids coming down the tracheal airway from all directions instead of from any particular position or side.

In select embodiments, the cuffs 110, 115 may be configured to form three-dimensional oblong-shapes, such as, strawberry, pear, or heart shaped, for example, having the inward funnel-shaped portions 175, 176. Cuffs 110, 115 may also be configured where the inward portion of the cuff is formed to work as a funnel to facilitate the capture of fluid drainage. First cuff 110 is disposed adjacent to the second cuff 115 in series to act as an additional line of defense from tracheal fluids or debris from entering a patient's lungs. First cuff 110 may be smaller than second cuff 115 to be less intrusive to the patient's larynx 183 (see FIG. 4). The first cuff 110 may also provide the advantage of reduced manufacturing costs.

The first cuff 110 may further be configured via their funneled shape (175, 176) to facilitate the drainage of any fluids that pass by or through the second cuff 115. First and second cuffs 110, 115 are configured to seal the trachea (airway), thereby minimizing fluid aspiration and securing the ETT 100 in place once inflated by first and second pilot balloons 135, 140. Further, such a configuration may help prevent or reduce incidences of self-extubation, when the ETT 100 is removed on its own due to not being properly secured within the patient.

In some embodiments, the second cuff 115 may be disposed approximately 2 to 3 centimeters (cm) apart from the first cuff 110 to allow for a gap at or near the larynx 183 (see FIG. 4) of the patient, thereby preventing or minimizing the cuffs 110, 115 from contacting or damaging the same. Thus, the second cuff 115 may serve as a first line of defense by collecting all fluids coming down the trachea, further sealing the tracheal airway (minimizing fluid aspiration) and further securing the ETT 100 in place. In other words, in certain embodiments the ETT 100 is configured to have the second cuff 115 disposed immediately above the larynx 183 while the first cuff 110 is disposed immediately below the larynx 183 to prevent bronchial intubation and inadvertent extubation.

Endotracheal tubes are typically made from different biomaterials, such as soft, flexible biomaterials. The most common biomaterial used to manufacture endotracheal tubes is polyvinyl chloride with an added plasticizer. Other synthetic materials are also currently used or being developed. In different embodiments different materials may be used to manufacture the ETT 100.

FIG. 2 is an enlarged view of the first evacuation port 150 and the first cuff 110 of the ETT apparatus 100 according to certain embodiments of the disclosure. In FIG. 2, the first evacuation port 150 is disposed proximal or at a bottom portion 174 of the funnel-shaped portion 175 of first cuff 110 where any draining fluids at 179 from the trachea and/or second cuff 115 would collect due to gravity and fluid flow principles. The bottom portion 174 of the funnel-shaped portion 175 is disposed in the direction and position nearest the beveled opening 170. The funnel-shaped portion 175 included in the configuration of the first cuff 110 allows for better drainage/collection of fluids flowing down the trachea in order to prevent or avoid fluid aspiration in the patient. The first evacuation port 150 having a configuration of a circumferential or 360° port provides an efficient manner in which to suction/collect the fluids captured by the funnel-shaped portion 175 of first cuff 110. First evacuation port 150 may be disposed proximal or at the bottom portion 174 of the funnel-shaped portion 175 of the first cuff 110 in order to better capture and suction these fluids. Further, the first evacuation port 150 is connected to evacuation channel 177 which in turn is in communication with evacuation lumen 120 which in turn is connected to a vacuum source (not shown) to suction any collected fluids/debris in the funnel-shaped portion 175.

FIG. 3 is an enlarged view of the second evacuation port 155 and the second cuff 115 of the ETT apparatus 100 according to certain embodiments of the disclosure. In a similar manner as discussed with regard to FIG. 2, in FIG. 3, the second evacuation port 155 is disposed proximal to a bottom portion 173 of the funnel-shaped portion 176 of second cuff 115 where any draining fluids (at 179 in FIG. 2) from the trachea would collect due to gravity and fluid flow principles. The bottom portion 173 of the funnel-shaped portion 176 is disposed in the direction and position nearest the beveled opening 170. The funnel-shaped portion 176 included in the configuration of the second cuff 115 allows for better drainage/collection of fluids flowing down the trachea in order to prevent or avoid fluid aspiration in the patient. The second evacuation port 155 having a configuration of a circumferential or 360° port provides an efficient manner in which to suction/collect the fluids captured by the funnel-shaped portion 176 of the second cuff 115. Second evacuation port 155 may be disposed proximal or at the bottom portion of the funnel-shaped portion 176 of the second cuff 115 in order to better capture and suction these fluids. Further, the second evacuation port 155 is also connected to evacuation channel 177 which in turn is in communication with evacuation lumen 120 which in turn is connected to a vacuum source (not shown) to suction any collected fluids/debris in the funnel-shaped portion 176.

The funnel-shaped portions 175, 176 may be sized to accommodate various amounts of fluid/debris capture. In other words, funnel-shaped portion 175 of first cuff 110 may be smaller than the funnel-shaped portion 176 of the second cuff 115 since the ETT 100 is configured to have cuff 115 to initially capture fluids/debris from the patient's trachea with cuff 110 providing additional protection advantages as discussed above.

FIG. 4 is an illustrative view of the ETT apparatus 110 inserted via the mouth and through the trachea of a patient 180 according to certain embodiments of the disclosure. In FIG. 4, ETT 100, for example, is inserted through the mouth down through the trachea of patient 180. In this example, the ETT 100 enters down the tracheal airway or larynx 183 of the patient 180 such that a safety marking (not shown) is adapted for alignment next to the vocal cords of the patient 180, so that the depth of the ETT 100 is of a suitable depth according to standards followed by those skilled in the art so that the beveled opening 170 of the ETT 100 is in proper position for patient 180 to respirate. A securing device 185 may optionally be used to hold ETT 100 at an optimal position. An ETT adapter 195 may optionally be used at the proximal end 195 of the tracheal tube 105.

In preparation, a medical professional may perform a safety and functionality check of the ETT 100 by first assembling ETT 100 and connecting each lumen (120, 125, 130) to its respective chemical injection input or suction output. Next, the first and second cuffs 110, 115 are inflated and deflated via the first and second pilot balloons 135,140 to test their functionality. Next, the patency of the tracheal tube 105 is confirmed prior to use. Performing this safety and functionality check prior to use may avoid problems during a medical procedure which could cause harm to a patient.

In operation, the ETT 100 may be used by a medical professional by inserting a stylet into the ETT 100 to assist in controlling and moving the ETT 100 during the insertion process within a patient's trachea. Next, the medical professional may apply a lubrication gel onto the lower portion of the ETT 100. Once the ETT 100 preparation is completed, the medical professional prepares the patient 180 for insertion of the ETT 100. The patient 180 must be in a position where the patient 180 lies on their back and their neck is slightly extended with the nose pointing outwards. Next, the medical professional moves the patient's tongue using suitable pieces of equipment to visualize the patient's larynx 183. Now the medical professional inserts the ETT 100 into the larynx 183, such that safety markings (not shown) align next to the vocal cords of the patient 180. Then, the medical professional inflates the first and second cuffs 110, 115, respectively using the pilot balloons 135,140 associated with each cuff. Next, the medical professional ventilates the patient 180 and inspects at the patient's chest to verify the proper placement of the ETT 100 within the trachea. If the placement is improper, adjustments are made by the medical professional. Then, the medical professional secures the ETT 100 in place via securing means 185 and proceeds to connect the evacuating lumen 120, to a vacuum source (not shown). Next, the medical professional may perform a chest x-ray (CXR) as would be understood by those skilled in the art to verify the depth and position of the ETT 100 inside the patient's chest. Then, if the depth is not sufficient, the medical professional adjusts the ETT 100 to adjust its depth in the patient's chest, otherwise, the ETT 100 is placed into operation for patient 180.

As discussed above, in some embodiments, the ETT 100 provides the advantage of a dual line of defense, namely, the first and second cuffs 110, 115, in that, if part of either cuff 110, 115 was to rupture or fail during use, the remaining cuff may continue to perform its protective functions without the need of stopping the medical procedure and replacing the ETT 100.

In some embodiments of this disclosure, the ETT 100 may be configured for use with any suitable patient. A suitable patient may include either humans or animals of different sizes. The ETT 100 may be configured for single or multiple use, depending on the requirements of any particular patient.

In certain embodiments, color-coding is used to minimize or prevent confusion. For example, the pilot balloons 135, 140 may be a specified color, such as light blue and labelled “U” for the upper or second cuff 115 and labelled “L” for the lower or first cuff 110. In another example, the injection lumens 125, 130 may be a specific color, such as green and labelled “R” for the right or first injection lumen 125 and labelled “L” for the left or second injection lumen 130. Further, the evacuation lumen 120 may be a specified color such as yellow for distinction.

The present disclosure includes the advantage of utilizing two cuffs disposed in series which provide an improved configuration which anchors the ETT 100 at positions immediately above and below the larynx 183, thereby preventing bronchial intubation and inadvertent extubation. The present disclosure also includes the advantage of eliminating aspiration by virtue of placing the second cuff 115 immediately above the larynx 183 to keep the upper airway secretions from entering the laryngeal area. After insertion and initial inflation of the cuffs 110, 115, the second cuff 115 rests above the larynx 183 thereby preventing exposure of the larynx 183 and trachea to contamination. By having the inflated cuffs 110, 115 described herein, oral secretions may be captured and removed by a suction catheter connected to the evacuation lumen 120 resulting in protecting both the larynx 183 and trachea during the use of ETT 100 and its subsequent removal from the patient 180. In other words, any secretions or fluids or debris is successfully removed via suction, thereby minimizing or eliminating such secretions or fluids or debris from entering the patient's lungs causing VAP or the like.

Thus, the disclosed dual cuff (110, 115) configuration is superior to conventional single cuffs by (1) providing a positive means of anchoring the cuff between the vocal cords, thereby preventing tube motion, accidental extubation, or further penetration of the ETT 100 into a bronchus, (2) lowering sealing pressure within the trachea and above the larynx 183, thereby reducing the risk of damaging the trachea or larynx 183, and (3) excluding secretions from the larynx 183 or upper trachea.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, define, in part, the scope of the foregoing claim terminology such that no inventive subject matter is dedicated to the public.

1. An endotracheal tube, comprising:
a tracheal tube having a proximal end and a distal end;
a plurality of spaced-apart cuffs disposed around the tracheal tube, the plurality of spaced-apart cuffs being configured to seal a tracheal airway of a patient and to capture tracheal secretions or debris;
at least two pilot balloons connected to the plurality of spaced-apart cuffs, the at least two pilot balloons being configured to inflate and deflate the plurality of spaced-apart cuffs;
at least two injection lumens connected to at least two injection distal ports and being configured to administer fluids to the bronchi of the patient; and
at least one evacuation lumen connected to a plurality of evacuation ports, the at least one evacuation lumen being connected to a vacuum source,
wherein the plurality of evacuation ports is disposed proximal the plurality of spaced-apart cuffs to remove the captured fluids or debris.

2. The endotracheal tube according to claim 1, wherein the tracheal tube includes an internal lining disposed on an inner wall of the tracheal tube and being configured to suppress growth of pathogens.

3. The endotracheal tube according to claim 2, wherein the internal lining is comprised of copper.

4. The endotracheal tube according to claim 2, wherein the internal lining is further comprised of at least one of brass, bronze, cupronickel, and copper-nickel-zinc.

5. The endotracheal tube according to claim 3, wherein the internal lining is spiral-shaped and configured to support the structure of the tracheal tube while maintaining the patency of the tracheal tube.

6. The endotracheal tube according to claim 3, wherein the internal lining is sinuous-shaped and configured to support the structure of the tracheal tube while maintaining the patency of the tracheal tube

7. The endotracheal tube according to claim 1, wherein the plurality of cuffs include
a first cuff disposed below a larynx and configured to seal the tracheal airway and secure the tracheal tube in position within the trachea; and
a second cuff disposed above the larynx and configured to seal the tracheal airway and secure the tracheal tube in position within the trachea.

8. The endotracheal tube according to claim 7, wherein the first cuff, when inflated is a three-dimensional oblong-shape having a funnel-shaped portion configured to capture tracheal fluids or debris, and
wherein the second cuff, when inflated is a three-dimensional oblong-shape having a funnel-shaped portion configured to capture tracheal fluids or debris.

9. The endotracheal tube according to claim 1, the plurality of evacuation ports includes
a first circumferential 360° evacuation port configured to collect fluids or debris from all directions; and
a second circumferential 360° evacuation port configured to collect fluids from all directions.

10. The endotracheal tube according to claim 9, wherein the first and the second circumferential 360° evacuation ports are connected to the at least one evacuation lumen.

11. The endotracheal tube according to claim 1, wherein the at least two injection lumens are configured to operate independently of each other to administer fluids directly to one and/or both bronchi.

12. The endotracheal tube according to claim 1, wherein each of the at least two pilot balloons are further configured to control and assess the amount of pressure within the plurality of spaced-apart cuffs.

13. The endotracheal tube according to claim 7, wherein the first cuff, when inflated is strawberry shaped having a funnel-shaped portion configured to capture tracheal fluids or debris, and
wherein the second cuff, when inflated is strawberry shaped having a funnel-shaped portion configured to capture tracheal fluids or debris.

14. The endotracheal tube according to claim 1, wherein the plurality of cuffs include
a first cuff disposed proximal the proximal end of the tracheal tube and configured to seal the tracheal airway and secure the tracheal tube in position within the trachea; and
a second cuff disposed proximal the distal end of the tracheal tube and configured to seal the tracheal airway and secure the tracheal tube in position within the trachea.

15. The endotracheal tube according to claim 7, wherein the first cuff is configured to be sized smaller than the second cuff.

16. The endotracheal tube according to claim 7, wherein the second cuff is disposed approximately 2 to 3 centimeters (cm) apart from the first cuff to allow for a gap at or near the larynx, thereby preventing or minimizing the first or second cuff from contacting or damaging the larynx upon insertion of the tracheal tube.

17. An endotracheal tube, comprising:
means for intubating a patient via their trachea;
means for sealing a tracheal airway of the patient and for capturing tracheal secretions or debris within the patient;
means for inflating and deflating the means for sealing and capturing;
means for administering fluids to the bronchi of the patient; and
means for evacuating the captured tracheal secretions or debris,
wherein the means for evacuating is disposed proximal the means for capturing tracheal secretions or debris.
